(19) United States
(12) Patent Application Publication
(10) Pub. No.: US 2006/0206165 A1
(43) Pub. Date: Sep. 14, 2006

(54) Occipital nerve stimulation to treat headaches and other conditions

(76) Inventors: Kristen N. Jaax, Saugus, CA (US); Todd K. Whitehurst, Santa Clarita, CA (US); Rafael Carbunaru, Studio City, CA (US); James C. Makous, Santa Clarita, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/256,356

(22) Filed: Oct. 21, 2005

Related U.S. Application Data
(60) Provisional application No. 60/661,700, filed on Mar. 14, 2005.

(51) Int. Cl. A61N 1/34

(52) U.S. Cl. 607/46

(57) ABSTRACT

A method of treating headaches includes stimulating a nerve in a patient's head with an electrode implanted over the skull on a posterior or superior portion of the patient's head to alleviate headache pain.

### RELATED APPLICATIONS

The present application claims the priority under 35 U.S.C. § 119 (e) of previous U.S. Provisional Patent Application No. 60/661,700 filed Mar. 14, 2005 for “Headache Treatment.” This provisional application is hereby incorporated by reference in its entirety.

### BACKGROUND

The public health significance of many medical, psychiatric, and neurological conditions and/or disorders is often overlooked, probably because of their episodic nature and the lack of mortality attributed to them. However, some medical conditions, such as headaches and facial pain, are often incapacitating, with considerable impact on social activities and work, and may lead to the significant consumption of drugs.

Migraine headaches are a particular form of headache, usually very intense and disabling. Migraines are a neurological disease thought to be of vascular origin. Migraines are characterized by attacks of sharp pain usually involving one half of the skull and accompanied by nausea, vomiting, phonophobia, photophobia and occasionally visual, olfactory or balance disturbances known as aura. The symptoms and their timing vary considerably among migraine sufferers and, to a lesser extent, from one migraine attack to the next. Migraine is often connected with the expansion of the blood vessels of the head and neck.

Migraine headaches can accompany, or be confused with, other types of headache, such as tension headache. Since the treatment for other forms of headache may differ from that for migraine, it is important to recognize when migraine, tension or other forms of headache are occurring. In some cases, migraine headaches can cause seizures. Additionally, stroke symptoms (passing or permanent) are seen in very severe subtypes.

Migraine headaches often run in families and frequently start in adolescence, although some research indicates that it can start in early childhood or even in utero. Migraines occur more frequently in women than men, and are most common between ages 15-45, with the frequency of attacks declining with age in most cases. Because their symptoms vary, an intense headache may be misdiagnosed as a migraine by a layperson.

Conventional treatments for migraines focus on three areas: trigger avoidance, symptomatic control, and preventive drugs. Each of these will be discussed below.

In a minority of patients, the incidence of migraine can be reduced through diet changes to avoid certain chemicals that serve as a trigger for the migraine. These chemical triggers may be present in such foods as cheddar cheese and chocolate, and in most alcoholic beverages. Other triggers may be situational and can be avoided through lifestyle changes. Such triggers may include particular points in the menstrual cycle, certain weather patterns, or hunger. Bright flashing lights may also be a trigger. Most migraine sufferers are sensitive to and avoid bright or flickering lights.

If a migraine occurs despite trigger avoidance, the next step in treatment is symptomatic control. Caffeine and simple pain killers, analgesics, such as paracetamol, aspirin or low doses of codeine are sometimes, but not often, effective. Anti-emetics by suppository or injection may be needed in cases where vomiting dominates the symptoms. Generally, the earlier these drugs are taken in the attack, the better their effect. Narcotic pain medications, such as heroin, morphine, and other opiates, provide variable relief. However, their side effects and ability to cause serious drug addiction contraindicates their general use.

Sumatriptan (Imitrex®) and the related 5-hydroxytryptamine (serotonin) receptor agonists are now available and are the therapy of choice for severe migraine attacks. They are highly effective, reducing or abolishing all the symptoms within 30 to 90 minutes. These drugs have few side effects if used in correct dosage and frequency. However, about 20-30% of patients do not respond.

Evidence is accumulating that these drugs are effective because they constrict certain blood vessels in the brain. They do this by acting at serotonin receptors on nerve endings. This action leads to a decrease in the release of a peptide known as CGRP. In a migraine attack, this peptide is released and may produce pain by dilating cerebral blood vessels.

In addition to treating symptoms, preventive medication may also be administered on a daily basis if attacks occur more often than every two weeks. A large number of preventative medications with varying modes of action can be used. Selection of a suitable medication for any particular patient is a matter of trial and error, since the effectiveness of individual medications varies widely from one patient to the next. Beta blockers such as propranolol and atenolol are usually tried first. Antidepressants such as amitriptyline may be effective. Antispasmodic drugs are used less frequently. Sansert was effective in many cases, but has been withdrawn from the U.S. market.

Migraine sufferers also usually develop their own coping mechanisms for intractable pain. A cold or hot shower directed at the head, less often a warm bath, or resting in a dark and silent room may be as helpful as medication for many patients.

### SUMMARY

Methods of treating chronic headaches, particularly migraine headaches, include stimulating a nerve in a patient's head with an electrode implanted over the skull on a posterior or superior portion of the patient's head to alleviate headache pain. Optimal placement of an implanted electrode or stimulator along the back or posterior part of the patient's head or on the top or superior portion of the patient's skull in an area of relatively low fat and soft tissue content minimizes the impendence presented to the stimulator and so minimizes the recharging cycle.

### BRIEF DESCRIPTION OF THE DRAWINGS

The accompanying drawings illustrate various embodiments of the present invention and are a part of the specification. The illustrated embodiments are merely examples of the present invention and do not limit the scope of the claims.

FIG. 1 illustrates an exemplary stimulator or system control unit (SCU) that may be used to apply stimulation to a stimulation site such, as the occipital nerves, to treat headaches and other conditions according to principles described herein.

FIG. 2 illustrates an exemplary microstimulator that may be used as an SCU to apply stimulation to a stimulation site, such as the occipital nerves, to treat headaches and other conditions according to principles described herein.

FIG. 3 shows one or more catheters coupled to the microstimulator according to principles described herein.

FIG. 4 depicts a number of SCUs configured to communicate with each other and/or with one or more external devices according to principles described

FIG. 5 illustrates the occipital nerves at the back of a human head and further illustrates optimal implantation sites for a stimulator stimulating the occipital nerves according to principles described herein.

FIG. 6 illustrates the location of the major nerves and arteries in the human head as viewed from above.

FIG. 7 shows an SCU implanted in the skull relatively close to the top or superior portion of the head so as to stimulate the occipital nerves to treat headaches and other conditions according to principles described herein.

Throughout the drawings, identical reference numbers designate similar, but not necessarily identical, elements.

### DETAILED DESCRIPTION

It has been discovered that stimulating one or more of the nerves in the head with an electrical stimulation current can alleviate or eliminate headache pain for patients who do not respond to other forms of treatment or who do not prefer any of the other forms of treatment. This includes migraine headaches.

Consequently, a stimulator may be implanted in a patient to deliver an electrical stimulation current to one or more of the nerves in the head, particularly the occipital nerves. This stimulation may be effective to treat headache pain and other types of pain or conditions, such as occipital neuralgia, facial pain, etc. The present specification will describe methods of implanting such a stimulator to most conveniently treat a variety of conditions, particularly headaches.

As used herein, and in the appended claims, the term “stimulator” will be used broadly to refer any device that delivers a stimulus, such as an electrical stimulation current or one or more drugs. Thus, the term “stimulator” includes, but is not limited to, a stimulator, microstimulator, implantable pulse generator (IPG), stimulation control unit (SCU) or similar device. As used herein and in the appended claims, the terms “stimulator” and “SCU” will be used interchangeable to refer to any implantable device that delivers an electrical stimulation current and, in some cases, drug stimulation. Implantable stimulators, also known as BION® devices (where BION® is a registered trademark of Advanced Bionics Corporation, of Valencia, Calif.), are typically characterized by a small, cylindrical housing which contains electronic circuitry that produces electric currents between spaced electrodes.

Electrical stimulation, also known as neuromodulation, will be described in more detail below. The implanted stimulator delivers an electrical current to a site on or near a target nerve or other tissue, e.g., the occipital nerves. This stimulation generally creates a tingling sensation, known as paresthesia, throughout a particular region of the body associated with the stimulated nerve. The size, intensity and character of the paresthesia may be controlled by adjusting the parameters of the stimulating current as will be described in more detail below.

The stimulus may additionally or alternatively include drug stimulation, also referred to herein as drug infusion. As will be described in more detail below, therapeutic dosages of one or more drugs may be infused into a stimulation site or into a site near the stimulation site. Additionally or alternatively, the stimulus applied to the stimulation site may include any other suitable stimulus such as, but not limited to, chemical stimulation, thermal stimulation, electromagnetic stimulation, and/or mechanical stimulation.

Some patients receive a stimulator to control or mask chronic pain associated with a particular nerve. However, it has been discovered that by stimulating one or more of the nerves in the head, the effect may be both a paresthesia in the region of the stimulated nerve and, additionally, a reduction or elimination of headache pain, particularly migraine headache pain. This is true even if the pain is not located at, or necessarily associated with, the site of stimulation or the nerves stimulated.

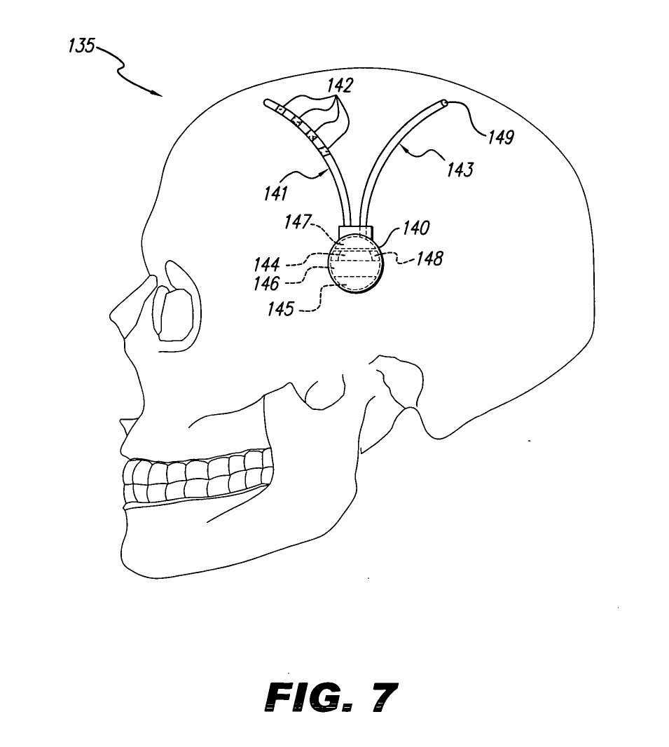

As will be described in more detail below, the implanted stimulator may be leaded or leadless. With a leaded stimulator, the stimulation pulse is delivered through a lead to an electrode or electrodes that are located on the lead that is connected to the implanted stimulator. This allows the body of the stimulator to be located at a convenient site remote from the nerve or other tissue being stimulated with the lead then delivering the stimulation current to the target site. An example of this is shown in FIG. 7 and described below. In other examples, the stimulator body or IPG may be located under the pectoral or arm, in the flank, abdomen, buttocks, head, etc. An indifferent electrode, also connected to the stimulator and/or at least one lead, completes the circuit, allowing the stimulation current to flow under control of the implanted stimulator. With a leadless stimulator, the electrodes are placed on the body of the stimulator and the stimulator is implanted at the site where the stimulation current is to be delivered.

The stimulating current that is output by an implanted stimulator is not constant, but is delivered in a regular cycle. Consequently, there are a number of parameters that characterize the current that is output by the implanted stimulator. As noted above, the effect of the stimulation can be controlled by adjusting these parameters of the stimulation current. For example, the size, intensity and character of the paresthesia created (and/or the location and/or amount of pain relief) can be controlled by adjusting the amplitude, frequency, pulse width, duty cycle, ramp up time, ramp down time and other parameters of the stimulation current. These parameters can be adjusted to tailor the stimulation to the needs of a particular patient.

These parameters can be adjusted over various ranges in a process called e-trolling, or current steering, to determine the best result for a patient. Manual trolling by manipulating the location of the electrode or electrodes relative to the stimulation site may also be tried. Different sets or programs of stimulation current parameters may be applied at different times or to different nerves to optimize the relief from headache pain afforded to the patient. Extremely low stimulation current frequencies, for example 2 Hz, may be effective to treat headache pain and other conditions, with a stimulator stimulating any one or more of the nerves in the head mentioned herein. A range of stimulation frequencies includes 2-150 Hz or more. A useful pulse width for a stimulation current may be 50-1500 microseconds

A stimulator or SCU may be implanted via injection and/or via endoscopic means adjacent to one or more of target stimulation sites. In some instances, however, a more complicated surgical procedure may be required for sufficient access to the target site and/or for purposes of fixing the SCU in place.

The following listed patents describe various details associated with the manufacture, operation, and use of BION implantable microstimulators, and are all incorporated herein by reference in their respective entireties:

| Application/ | Filing/ | |
|---|---|---|
| Patent/ | Publication | |
| Publication No. | Date | Title |
| U.S. Pat. No. | Issued | Implantable Microstimulator |
| 5,193,539 | Mar 16, 1993 | |
| U.S. Pat. No. | Issued | Structure and Method of Manufacture |
| 5,193,540 | Mar 16, 1993 | of an Implantable Microstimulator |
| U.S. Pat. No. | Issued | Implantable Device Having an |
| 5,312,439 | May 17, 1994 | Electrolytic Storage Electrode |
| U.S. Pat. No. | Issued | Battery-Powered Patient Implantable |
| 6,185,452 | Feb. 6, 2001 | Device |
| U.S. Pat. No. | Issued | System of Implantable Devices For |
| 6,164,284 | Dec. 26, 2000 | Monitoring and/or Affecting Body |
| | | Parameters |
| U.S. Pat. No. | Issued | System of Implantable Devices For |
| 6,208,894 | Mar. 27, 2001 | Monitoring and/or Affecting Body |
| | | Parameters |
| U.S. Pat. No. | Issued | Implantable Microstimulator and |
| 6,051,017 | Apr. 18, 2000 | Systems Employing Same |

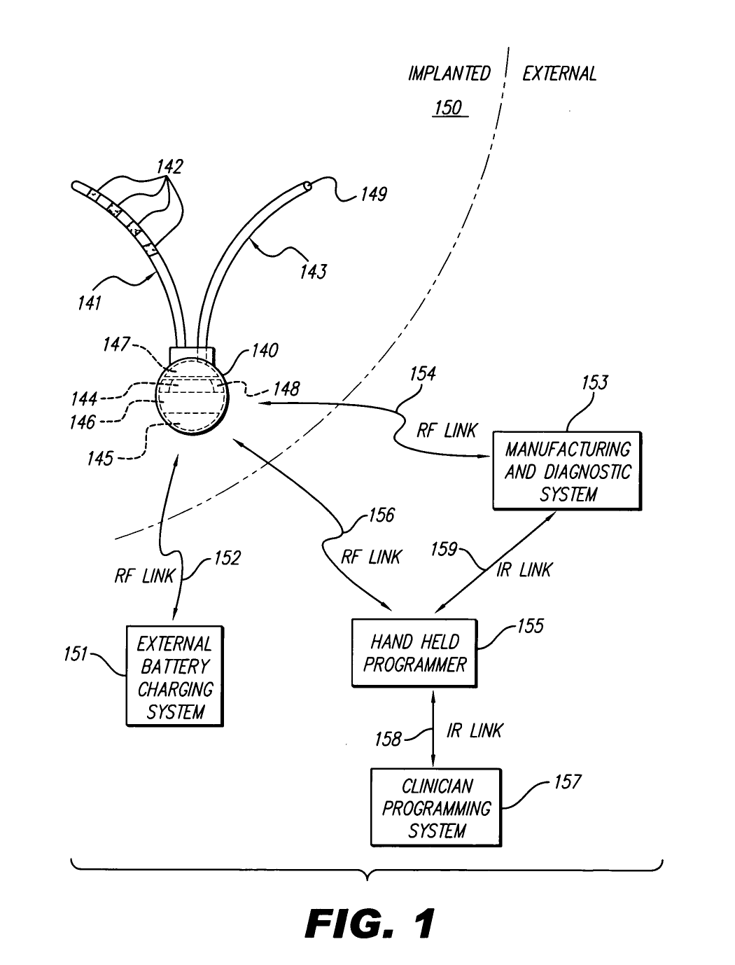

To facilitate an understanding of the methods of optimally placing a stimulator to treat headache pain and other conditions, as described herein, a more detailed description of the stimulator and its operation will now be given with reference to the figures. FIG. 1 illustrates an exemplary stimulator or SCU (140) that may be implanted within a patient (150) and used to apply a stimulus to a stimulation site, e.g., an electrical stimulation of the stimulation site, an infusion of one or more drugs into the stimulation site, or both. The electrical stimulation function of the SCU (140) will be described first, followed by an explanation of the drug delivery function of the SCU (140). It will be understood, however, that the SCU (140) may be configured to provide any type of stimulation as best suits a particular patient.

The exemplary SCU (140) shown in FIG. 1 is configured to provide electrical stimulation to a patient and includes a lead (141) having a proximal end coupled to the body of the SCU (140). The lead (141) also includes a number of electrodes (142) configured to apply an electrical stimulation current to a stimulation site. In some embodiments, the lead (141) includes anywhere between two and sixteen electrodes (142). However, the lead (141) may include any number of electrodes (142) as best serves a particular application. The electrodes (142) may be arranged as an array, for example, having at least two or at least four collinear electrodes. In some embodiments, the electrodes are alternatively inductively coupled to the SCU (140). The lead (141) may be thin (e.g., less than 3 millimeters in diameter) such that the lead (141) may be positioned near a stimulation site. Alternatively, as will be described in more detail below, the SCU (140) may be leadless.

As illustrated in FIG. 1, the SCU (140) includes a number of components. It will be recognized that the SCU (140) may include additional and/or alternative components as best serves a particular application. A power source (145) is configured to output voltage used to supply the various components within the SCU (140) with power and/or to generate the power used for electrical stimulation. The power source (145) may be a primary battery, a rechargeable battery, super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like. Alternatively, the SCU (140) may include one or more components configured to receive power from another medical device that is implanted within the patient.

When the power source (145) is a battery, it may be a lithium-ion battery or other suitable type of battery. When the power source (145) is a rechargeable battery, it may be recharged from an external system through a power link such as a radio frequency (RF) power link. One type of rechargeable battery that may be used is described in International Publication WO 01/82398 A1, published Nov. 1, 2001, and/or WO 03/005465 A1, published Jan. 16, 2003, both of which are incorporated herein by reference in their entireties. Other battery construction techniques that may be used to make a power source (145) include those shown, e.g., in U.S. Pat. Nos. 6,280,873; 6,458,171, and U.S. Application Publication Nos. 2001/0046625 A1and 2001/0053476 A1, all of which are incorporated herein by reference in their respective entireties. Recharging can be performed using an external charger.

The SCU (140) may also include a coil (148) configured to receive and/or emit a magnetic field (also referred to as a radio frequency (RF) field) that is used to communicate with or receive power from one or more external devices (151, 153, 155). Such communication and/or power transfer may include, but is not limited to, transcutaneously receiving data from the external device, transmitting data to the external device, and/or receiving power from the external device that is used to recharge the power source (145).

For example, an external battery charging system (EBCS) (151) may provide power used to recharge the power source (145) via an RF link (152). External devices including, but not limited to, a hand held programmer (HHP) (155), clinician programming system (CPS) (157), and/or a manufacturing and diagnostic system (MDS) (153), may be configured to activate, deactivate, program, and test the SCU (140) via one or more links (154, 156). It will be recognized that the links, which are RF links (152, 154, 156) in the illustrated example, may be any type of link used to transmit data or energy, such as an optical link, a thermal link, or any other energy-coupling link. One or more of these external devices (153, 155, 157) may also be used to control the infusion of one or more drugs by the SCU (140) into a stimulation site to stimulate the occipital nerves and other target sites to treat headaches and other conditions.

Additionally, if multiple external devices are used in the treatment of a patient, there may be some communication among those external devices, as well as with the implanted SCU (140). Again, any type of link for transmitting data or energy may be used among the various devices illustrated. For example, the CPS (157) may communicate with the HHP (155) via an infrared (IR) link (158), with the MDS (153) via an IR link (161), and/or directly with the SCU (140) via an RF link (160). As indicated, these communication links (158, 161, 160) are not necessarily limited to IR and RF links and may include any other type of communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the SCU (140). Furthermore, it will be recognized that the functions performed by any two or more of the HHP (155), MDS (153), CPS (157), and EBCS (151) may be performed by a single external device. One or more of the external devices (153, 155, 157) may be embedded in a seat cushion, mattress cover, pillow, garment, belt, strap, pouch, or the like so as to be positioned near the implanted SCU (140) when in use.

The SCU (140) may also include electrical circuitry (144) configured to produce electrical stimulation pulses that are delivered to the stimulation site via the electrodes (142). In some embodiments, the SCU (140) may be configured to produce monopolar stimulation. The SCU (140) may alternatively or additionally be configured to produce bipolar or tripolar stimulation. Monopolar electrical stimulation is achieved, for example, using the stimulator case as an indifferent electrode. Bipolar or tripolar electrical stimulation is achieved, for example, using one or more of the electrodes of the electrode array as an indifferent electrode. The electrical circuitry (144) may include one or more processors configured to decode stimulation parameters and generate the stimulation pulses. In some embodiments, the SCU (140) has at least four channels and drives up to sixteen electrodes or more. The electrical circuitry (144) may include additional circuitry such as capacitors, integrated circuits, resistors, coils, and the like configured to perform a variety of functions as best serves a particular application.

The SCU (140) may also include a programmable memory unit (146) for storing one or more sets of data and/or stimulation parameters. The stimulation parameters may include, but are not limited to, electrical stimulation parameters, drug stimulation parameters, and other types of stimulation parameters. The programmable memory (146) allows a patient, clinician, or other user of the SCU (140) to adjust the stimulation parameters such that the stimulation applied by the SCU (140) is safe and efficacious for treatment of a particular patient with chronic headaches or other condition being treated. The different types of stimulation parameters (e.g., electrical stimulation parameters and drug stimulation parameters) may be controlled independently. However, in some instances, the different types of stimulation parameters are coupled. For example, electrical stimulation may be programmed to occur only during drug stimulation. Alternatively, the different types of stimulation may be applied at different times or with only some overlap. The programmable memory (146) may be any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, burst pattern (e.g., burst on time and burst off time), duty cycle or burst repeat interval, ramp on time, and ramp off time of the stimulation current that is applied to the stimulation site. The drug stimulation parameters may control various parameters including, but not limited to, the amount of drugs infused into the stimulation site, the rate of drug infusion, and the frequency of drug infusion. For example, the drug stimulation parameters may cause the drug infusion rate to be intermittent, constant, or bolus. Other stimulation parameters that characterize other classes of stimuli are possible. For example, when tissue is stimulated using electromagnetic radiation, the stimulation parameters may characterize the intensity, wavelength, and timing of the electromagnetic radiation stimuli. When tissue is stimulated using mechanical stimuli, the stimulation parameters may characterize the pressure, displacement, frequency, and timing of the mechanical stimuli.

Specific stimulation parameters may have different effects on neural or other tissue. Thus, in some embodiments, the stimulation parameters may be adjusted by the patient, a clinician, or other user of the SCU (140) as best serves a particular stimulation site. The stimulation parameters may also be automatically adjusted by the SCU (140), as will be described below. For example, the amplitude of the stimulation current applied to a stimulation site may be adjusted to have a relatively low value to target a nerve having relatively large diameter fibers. The SCU (140) may also, or alternatively, increase excitement of a stimulation site by applying a stimulation current having a relatively low frequency to the stimulation site (e.g., less than 100 Hz). The SCU (140) may also, or alternatively, decrease excitement of a stimulation site by applying a relatively high frequency to the stimulation site (e.g., greater than 100 Hz). The SCU (140) may also, or alternatively, be programmed to apply the stimulation current to a stimulation site intermittently or continuously.

Additionally, the exemplary SCU (140) shown in FIG. 1 is configured to provide drug stimulation to a patient, for example, a headache patient, by applying one or more drugs to a stimulation site. For this purpose, the SCU (140) includes a pump (147). The pump (147) is configured to store and dispense the one or more drugs, for example, through a catheter (143). The catheter (143) is coupled at a proximal end to the SCU (140) and may have an infusion outlet (149) for infusing the one or more drugs into a stimulation site. In some embodiments, the SCU (140) may include multiple catheters (143) and/or pumps (147) for storing and infusing dosages of the one or more drugs into the stimulation site or into multiple stimulation sites.

The pump (147) or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps. Another example is a micro-drug pump.

Exemplary pumps (147) or controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 5,057,318; 5,059,423; 5,112,614; 5,137,727; 5,234,692; 5,234,693; 5,728,396; 6,368,315 and the like. Additional exemplary drug pumps suitable for use as described herein include, but are not necessarily limited to, those disclosed in U.S. Pat. Nos. 4,562,751; 4,678,408; 4,685,903; 5,080,653; 5,097,122; 6,740,072; and 6,770,067. Exemplary micro-drug pumps suitable for use as described herein include, but are not necessarily limited to, those disclosed in U.S. Patent Nos. 5,234,692; 5,234,693; 5,728,396; 6,368,315; 6,666,845; and 6,620,151. All of these listed patents are incorporated herein by reference in their respective entireties.

The SCU (140) of FIG. 1 is illustrative of many types of SCUs that may be used to stimulate the occipital nerves and other target sites to treat headaches and other conditions. For example, the SCU (140) may include an implantable pulse generator (IPG) coupled to one or more leads having a number of electrodes, a spinal cord stimulator (SCS), a cochlear implant, a deep brain stimulator, a drug pump (mentioned previously), a micro-drug pump (mentioned previously), or any other type of implantable stimulator configured to deliver a stimulus to a stimulation site within a patient. Exemplary IPGs suitable for use as described herein include, but are not necessarily limited to, those disclosed in U.S. Pat. Nos. 6,381,496, 6,553,263; and 6,760,626. Exemplary spinal cord stimulators suitable for use as described herein include, but are not necessarily limited to, those disclosed in U.S. Pat. Nos. 5,501,703; 6,487,446; and 6,516,227. All of these listed patents are incorporated herein by reference in their respective entireties.

Alternatively, the SCU (140) may be or include an implantable microstimulator, such as a BION® microstimulator (Advanced Bionics® Corporation, Valencia, Calif.). Various details associated with the manufacture, operation, and use of BION implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

The SCU (140) may be implanted within the patient (150) using any suitable surgical procedure such as, but not limited to, injection, small incision, open placement, laparoscopy, or endoscopy. Exemplary methods of implanting a microstimulator, for example, are described in U.S. Pat. Nos. 5,193,539; 5,193,540; 5,312,439; 6,185,452; 6,164,284; 6,208,894; and 6,051,017. Exemplary methods of implanting an SCS, for example, are described in U.S. Pat. Nos. 5,501,703; 6,487,446; and 6,516,227. Exemplary methods of implanting a deep brain stimulator, for example, are described in U.S. Patent Nos. 5,938,688; 6,016,449; and 6,539,263. All of these listed patents are incorporated herein by reference in their respective entireties.

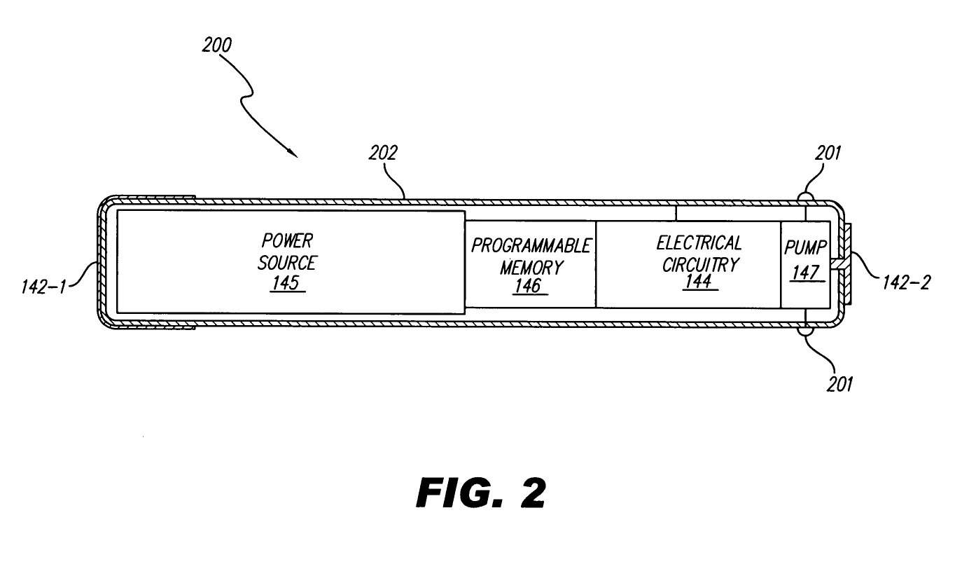

FIG. 2 illustrates an exemplary BION microstimulator (200) that may be used as the SCU (140; FIG. 1) described herein. Other configurations of the microstimulator (200) are possible, as shown in the above-referenced patents and as described further below.

As shown in FIG. 2, the microstimulator (200) may include the power source (145), the programmable memory (146), the electrical circuitry (144), and the pump (147) described above in connection with FIG. 1. These components are housed within a capsule (202). The capsule (202) may be a thin, elongated cylinder or any other shape as best serves a particular application. The shape of the capsule (202) may be determined by the structure of the desired stimulation site, the surrounding area, and/or the method of implantation. In some embodiments, the capsule (202) has a volume that is substantially equal to or less than three cubic centimeters.

In some embodiments, the microstimulator (200) may include two or more leadless electrodes (142). Either or both of the electrodes (142) may alternatively be located at the ends of short, flexible leads as described in U.S. patent application Ser. No. 09/624,130, filed July 24, 2000, which is incorporated herein by reference in its entirety. The use of such leads permits, among other things, electrical stimulation current to be directed more locally to targeted tissue(s) a short distance from the surgical fixation of the bulk of the microstimulator (200), while allowing most elements of the microstimulator (200) to be located in a more surgically convenient site. This minimizes the distance traversed and the surgical planes crossed by the microstimulator (200) and any lead(s).

The external surfaces of the microstimulator (200) may advantageously be composed of biocompatible materials. For example, the capsule (202) may be made of glass, ceramic, polymers, metal, or any other material that provides a hermetic package that will exclude water vapor but permit the passage of electromagnetic fields used to transmit data and/or power. The electrodes (142) may be made of a conducting ceramic, conducting polymer, and/or a noble or refractory metal, such as gold, silver, platinum, iridium, tantalum, titanium, titanium nitride, niobium or their alloys that are biocompatible, e.g., minimize corrosion, electrolysis, and damage to the surrounding tissues.

The microstimulator (200) may be implanted within a patient with a surgical tool such as a hypodermic needle, bore needle, or any other tool specially designed for the purpose. Alternatively, the microstimulator (200) may be implanted using endoscopic or laparoscopic techniques. The microstimulator (200) may also be implanted and, in some cases, fixed in place, through an incision. As previously mentioned, the microstimulator (200) may be coupled directly to a stimulation site.

FIG. 2 shows that the microstimulator (200) may also include one or more infusion outlets (201). The infusion outlets (201) facilitate the infusion of one or more drugs into a stimulation site to treat a particular medical condition. The infusion outlets (201) may dispense one or more drugs, chemicals, or other substances directly to the stimulation site. Alternatively, as will be described in more detail below, catheters may be coupled to the infusion outlets (201) to deliver the drug therapy to a stimulation site some distance from the body of the microstimulator (200). The stimulator (200) of FIG. 2 also includes electrodes (142-1 and 142-2) at either end of the capsule (202). One of the electrodes (142) may be designated as a stimulating electrode to be placed close to the stimulation site and one of the electrodes (142) may be designated as an indifferent electrode used to complete a stimulation circuit.

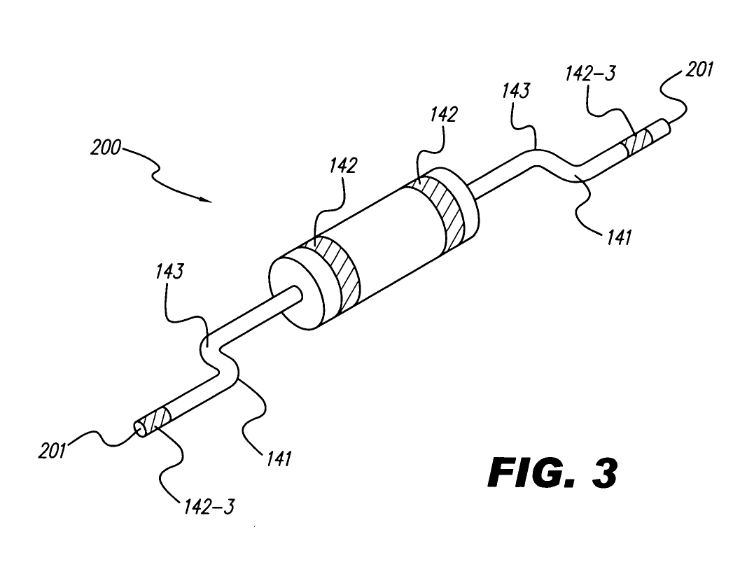

FIG. 3 shows an example of a microstimulator (200) with one or more catheters (143) coupled to the infusion outlets on the body of the microstimulator (200). With the catheters (143) in place, the infusion outlets (201) that actually deliver the drug therapy to target tissue are located at the ends of catheters (143). Thus, in the example of FIG. 3, a drug therapy is expelled by the pump (147, FIG. 2) from an infusion outlet (201, FIG. 2) in the casing (202, FIG. 2) of the microstimulator (200), through the catheter (143), out an infusion outlet (201) at the end of the catheter (143) to the stimulation site within the patient. As shown in FIG. 3, the catheters (143) may also serve as leads (141) having one or more electrodes (142-3) disposed thereon. Thus, the catheters (143) and leads (141) of FIG. 3 permit infused drugs and/or electrical stimulation current to be directed to a stimulation site while allowing most elements of the microstimulator (200) to be located in a more surgically convenient site. The example of FIG. 3 may also include leadless electrodes (142) disposed on the housing of the microstimulator (200), in the same manner described above.

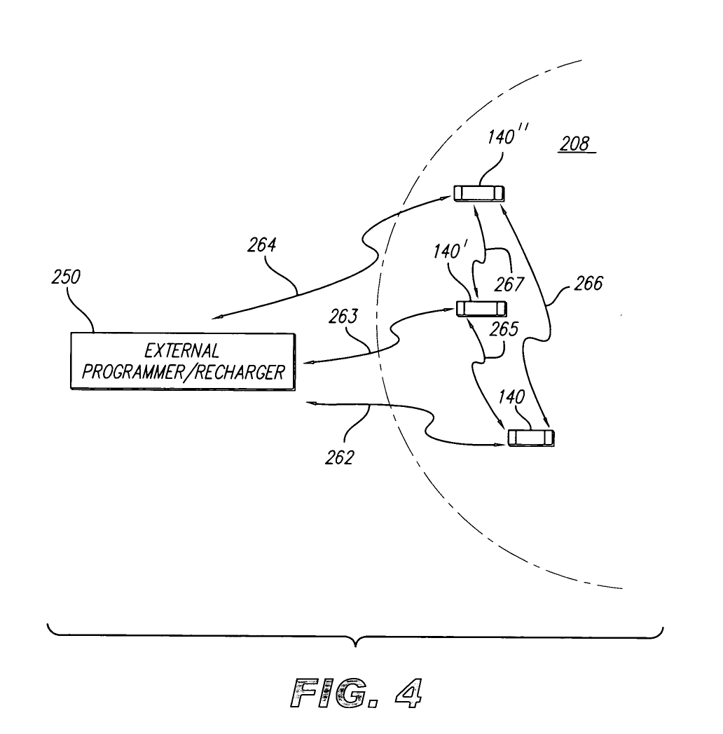

AN SCU may be configured to operate independently. Alternatively, as shown in FIG. 4 and described in more detail below, the SCU (140) may be configured to operate in a coordinated manner with one or more additional SCUs, other implanted devices, or other devices external to the patient's body. For instance, a first SCU may control or operate under the control of a second SCU, other implanted device, or other device external to the patient's body. The SCU (140) may be configured to communicate with other implanted SCUs, other implanted devices, or other devices external to the patient's body via an RF link, an untrasonic link, an optical link, or any other type of communication link. For example, the SCU (140) may be configured to communicate with an external remote control unit that is capable of sending commands and/or data to the SCU (140) and that is configured to receive commands and/or data from the SCU (140).

In order to determine the strength and/or duration of electrical stimulation and/or amount and/or type(s) of stimulating drug(s) required to most effectively treat a headache or other condition, various indicators of headache and/or a patient's response to treatment may be sensed or measured. These indicators include, but are not limited to, electrical activity of the brain (e.g., EEG); neurotransmitter levels; hormone levels; metabolic activity in the brain; blood flow rate in the head, neck or other areas of the body; medication levels within the patient; patient input, e.g. when prodromal symptoms are sensed the patient can push a button on a remote control or other external unit; temperature of tissue in stimulation target region, including the occipital nerve; physical activity level, e.g. based on accelerometer recordings; brain hyperexcitability, e.g. increased response of given tissue to the same input; indicators of collateral tissue stimulation might be used to adjust stimulation parameters; and/or detection of muscle tone in neck (mechanical strain, pressure sensor, EMG). In some embodiments, the SCU (140) may be configured to change the stimulation parameters in a closed loop manner in response to these measurements. The SCU (140) may be configured to perform the measurements. Alternatively, other sensing devices may be configured to perform the measurements and transmit the measured values to the SCU (140). Exemplary sensing devices include, but are not limited to, chemical sensors, electrodes, optical sensors, mechanical (e.g., motion, pressure) sensors, and temperature sensors.

Thus, one or more external appliances may be provided to interact with the SCU (140), and may be used to accomplish at least one or more of the following functions:

Function 1: If necessary, transmit electrical power to the SCU (140) in order to power the SCU (140) and/or recharge the power source (145).

Function 2: Transmit data to the SCU (140) in order to change the stimulation parameters used by the SCU (140).

Function 3: Receive data indicating the state of the SCU (140) (e.g., battery level, drug level, stimulation parameters, etc.).

Additional functions may include adjusting the stimulation parameters based on information sensed by the SCU (140) or by other sensing devices.

By way of example, an exemplary method of treating a patient with a chronic headache or other condition may be carried out according to the following sequence of procedures. The steps listed below may be modified, reordered, and/or added to as best serves a particular application.

1. An SCU (140) is implanted so that its electrodes (142) and/or infusion outlet (149) are coupled to or located near a stimulation site (e.g., the occipital nerves or other nerves in the patient's head). If the SCU (140) is a microstimulator, such as the BION microstimulator (200; FIG. 2), the microstimulator itself may be coupled to the stimulation site.

2. The SCU (140) is programmed to apply at least one stimulus to the stimulation site. The stimulus may include electrical stimulation, drug stimulation, chemical stimulation, thermal stimulation, electromagnetic stimulation, mechanical stimulation, and/or any other suitable stimulation.

3. When the patient desires to invoke stimulation, the patient sends a command to the SCU (140) (e.g., via a remote control) such that the SCU (140) delivers the prescribed stimulation. The SCU (140) may be alternatively or additionally configured to automatically apply the stimulation in response to sensed indicators of headache or other patient condition.

4. To cease stimulation, the patient may turn off the SCU (140) (e.g., via a remote control).

5. Periodically, the power source (145) of the SCU (140) is recharged, if necessary, in accordance with Function 1 described above. As will be described below, this recharging function can be made much more efficient using the principles disclosed herein.

In other examples, the treatment administered by the SCU (140), i.e., drug therapy and/or electrical stimulation, may be automatic and not controlled or invoked by the patient.

For the treatment of different patients with chronic headache or other conditions, it may be desirable to modify or adjust the algorithmic functions performed by the implanted and/or external components, as well as the surgical approaches. For example, in some situations, it may be desirable to employ more than one SCU (140), each of which could be separately controlled by means of a digital address. Multiple channels and/or multiple patterns of stimulation may thereby be used to deal with the various components of a headache condition, such as the combination of migraine with another form or forms of headache or the combination of headache with facial or other pain.

As shown in the example of FIG. 4, a first SCU (140) implanted beneath the skin of the patient (208) provides a stimulus to a first location; a second SCU (140′) provides a stimulus to a second location; and a third SCU (140″) provides a stimulus to a third location. As mentioned earlier, the implanted devices may operate independently or may operate in a coordinated manner with other implanted devices or other devices external to the patient's body. That is, an external controller (250) may be configured to control the operation of each of the implanted devices (140, 140′, and 140″). In some embodiments, an implanted device, e.g. SCU (140), may control or operate under the control of another implanted device(s), e.g. SCU (140′) and/or SCU (140″). Control lines (262-267) have been drawn in FIG. 4 to illustrate that the external controller (250) may communicate or provide power to any of the implanted devices (140, 140′, and 140″) and that each of the various implanted devices (140, 140′, and 140″) may communicate with and, in some instances, control any of the other implanted devices.

As a further example of multiple SCUs (140) operating in a coordinated manner, the first and second SCUs (140, 140′) of FIG. 4 may be configured to sense various indicators of a headache or other condition and transmit the measured information to the third SCU (140″). The third SCU (140″) may then use the measured information to adjust its stimulation parameters and apply stimulation to a stimulation site accordingly (e.g., to the occipital nerves). The various implanted SCUs may, in any combination, sense indicators of headache or other conditions, communicate or receive data on such indicators, and adjust stimulation parameters accordingly.

Alternatively, the external device (250) or other external devices communicating with the external device may be configured to sense various indicators of a patient's condition. The sensed indicators can then be collected by the external device (250) for relay to one or more of the implanted SCUs or may be transmitted directly to one or more of the implanted SCUs by any of an array of external sensing devices. In either case, the SCU, upon receiving the sensed indicator(s), may adjust stimulation parameters accordingly. In other examples, the external controller (250) may determine whether any change to stimulation parameters is needed based on the sensed indicators. The external device (250) may then signal a command to one or more of the SCUs to adjust stimulation parameters accordingly.

The nerve or nerves stimulated to treat headache pain include, for example, but are not limited to, any cranial nerve; the greater, lesser or third occipital nerves; the trigeminal nerve; the infraorbital nerve; the facial nerve; the maxillary nerve, the mandibular nerve and divisions of those nerves such as the two branches of the ophthalmic division of the trigeminal nerve, i.e., the supratrochlear and suprorbital nerves; the zygomaticotemporal nerve branching from the maxillary division of the trigeminal nerve: the auriculotemporal nerve branching from the mandibular division of the trigeminal nerve. However, stimulation of the occipital nerves has been shown to be particularly effective in treating chronic headache pain.

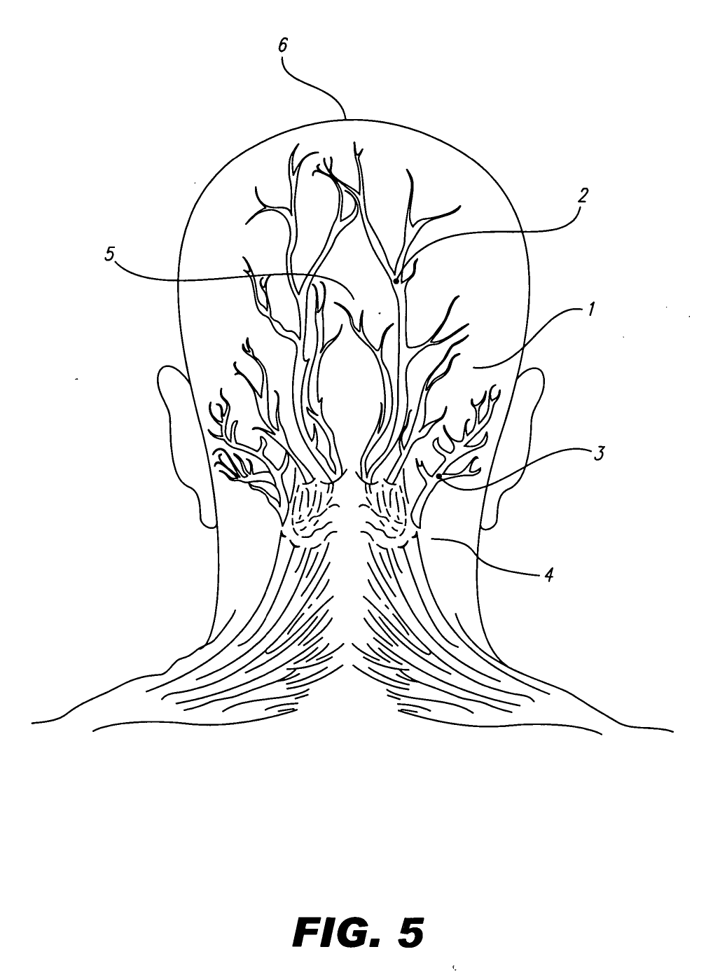

As shown in FIG. 5, the occipital nerves (1) originate in the neck and extend up the back of the head. The occipital nerve (1) is divided into greater (2) and lesser (3) occipital nerves.

As described above, implanting a stimulator to provide an electrical stimulation to the occipital nerves (1) has been known to create a paresthesia at the stimulation site. Consequently, such stimulation may be used to treat conditions such as occipital neuralgia as described in U.S. Patent No. 6,505,075 to Weiner, which is incorporated herein by reference in its entirety.

In addition to creating a local paresthesia, stimulation of the occipital nerves has also been shown to have a therapeutic effect on headache pain that may or may not have any demonstrable connection with the occipital nerves. This is true of both migraine and other forms of chronic headaches.

Typically, when stimulating the occipital nerves, either to treat occipital neuralgia, an implantable stimulator is implanted in the patient's neck at or near the base of the skull (4). While this is an effective placement to stimulate the occipital nerves and treat the various conditions described herein, there are also disadvantages.

The region (4) where stimulators have previously been implanted to stimulate the occipital nerves is an area with a relatively high content of fat and soft tissue. This fat and soft tissue present a low impedance to the electrical stimulation current output by the implanted stimulator. As a result, an increased amount of power is required to produce a current of sufficient amplitude in the volume of the stimulation site to create the desired paresthesia or otherwise stimulate the occipital nerves for treatment of the various conditions described herein and the like.

As a result of this increased power requirement, a non-rechargeable implant is depleted of power more quickly. Similarly, a rechargeable implanted stimulator must be recharged frequently.

As described above, recharging the implanted stimulator requires bringing the stimulator into proximity with an external device that can transmit power transcutaneously to the stimulator. As illustrated and explained in connection with FIG. 4, this requires the patient in whom the simulator is implanted to keep the stimulator in proximity to the external charging device during the recharging cycle. Typically, the external charging device is plugged into a wall outlet or similar power source and so requires the patient to stay in place, perhaps even in a particular position, during recharging.

Recharging the stimulator takes hours each time the stimulator is charged. Moreover, to allow the stimulator to operate effectively in the environment of fat and soft tissue found at the base of the skull (4), the patient may have to recharge the stimulator multiple times per day. Consequently, this potentially burdensome and lengthy charging routine can have a significant negative impact on the lives of patients who are using an implanted stimulator to treat chronic headache pain or other conditions treated by stimulation of the occipital nerves or other cranial nerves.

To alleviate this problem, it has been discovered that the implanted stimulator or stimulating electrode can be implanted higher on the skull than has traditionally been thought. For all occipital nerve stimulators located on the posterior scalp, the stimulator or stimulating electrode can be placed in the subcutaneous fat which lies just beneath the skin. The approximate depth of this implant is 2-5 mm below the skin. The greater occipital nerve is known to travel in this tissue plane, the subcutaneous fat, and therefore the electrodes placed in this tissue plane will typically be within 2-4 mm of the nerve. The exception, of course, is morbidly obese patients who may have exceptionally thick layers of subcutaneous fat in all regions of the body. The tissue environment at that depth is subcutaneous fat. This fat is bounded superficially by the skin. Below the subcutaneous fat lies the skull, although in certain regions there is a thin muscle that lies between the subcutaneous fat and the skull, called the occipital belly of the occipitofrontalis.

In the region of the trapezius, the tissue environment is similar in the superficial planes. The stimulator or stimulating electrode is implanted in the subcutaneous fat and the only tissue overlying that fat is skin. Below the subcutaneous fat in this region, however, lie several layers of muscle (ordered from most superficial to least superficial): the trapezius, the splenius capitis, the semispinalis capitis, the spinalis capitis, the rectus capitis posterior major, the rectus capitis posterior minor, the obliquus capitis superior, the rectus capitus lateralis. Below all of these muscles lie the vertebral bodies of C1 and C2.

Placing the electrode higher up under the scalp, as opposed to the C1 placement of an unleaded stimulator is likely to improve energy consumption in a number of ways. First, with scalp placement, there is higher overall impedance; the GON is located in a very small low-impedance subcutaneous space bounded by the high-impedance skull, scalp, and air above the scalp. In contrast, placement at C1 involves a much more substantial volume of low-impedance muscle, fatty tissue, and connective tissue in close proximity to the bion. Because electrical current dissipates through low-impedance tissue, the anatomy of the head will confine the stimulation to a region between the skull and the scalp. Less current will dissipate at the leaded electrode than at the C1 placement of the unleaded bion. Therefore, lower-amplitude stimulation will be sufficient for occipital nerve stimulation using a leaded bion.

As shown in FIG. 1, the stimulator can be implanted along the occipital nerves (1) at the back of the head (5) or on the top or superior portion of the skull (6). While such placement may not be effective to treat some forms of occipital neuralgia that need stimulation at a lower point along the occipital nerves (1), such placement is still effective to treat headache pain, including migraine headache pain, and may also be effective to treat other conditions such as some forms of occipital neuralgia and other conditions treated through stimulation of the occipital nerves or other cranial nerves.

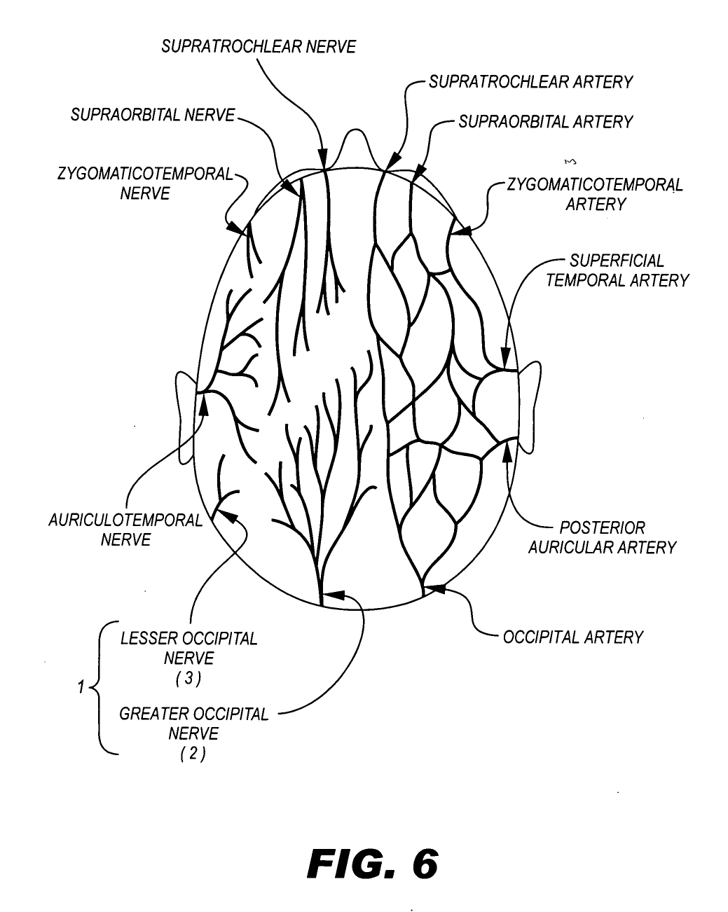

FIG. 6 illustrates a view of the major nerves and arteries in the human head as viewed from above looking down on the top or superior part of the head. As shown in FIG. 6, the greater occipital nerves (2) extend to and across some of the top or superior portion of the head. The Lesser occipital nerves (3) may also extend to or near the top or superior portion of the head. Consequently, as described above, an implanted stimulator can be located along the back or posterior part of the head or on the top or superior portion of the head and still provide a stimulus to the occipital nerves (1).

Returning to FIG. 5, the advantage of placing the implanted stimulator above the base of the skull (4), along the back or posterior portion of the head (5) or on the top or superior part of the head (6) is the reduced fat and soft tissue content in those areas as compared with the traditional implantation location (4) at the base of the skull. This reduced fat and soft tissue content allows the stimulator to use significantly less power. The lower power consumption results because there is less low-impedance fat and more high-impedance structures such as the skull and air (since the skin is thin here).

As a result, current need not be infused into such a large volume to achieve the amplitudes needed at the nerve being stimulated. The high impedance structures essentially guide the current along the subcutaneous fat to the nerve being stimulated. Thus, the current or field can be more accurate steered to the target tissue. Consequently, the implanted stimulator uses less power.

As the power consumption of the stimulator is decreased, a number of benefits are realized. For example, a non-rechargeable stimulator will have an increased operating life and a rechargeable stimulator will require much less frequent recharging. This will also result in fewer stimulator explants.

As the stimulator uses less power, the patient with a rechargeable stimulator has to spend less time recharging. As described herein, the patient may be restrained in his or her activity for significant amounts of time as a stimulator is recharged. The less the stimulator needs to be recharged, the more mobility and freedom the implant patient has with a decreased impact on the patient's lifestyle resulting from the implant.

Additionally, if the stimulator or stimulating electrode are located above the base of the skull (4), along the back or posterior portion of the head (5) or on the top or superior part of the head (6) where there is reduced fat and soft tissue content, there is also less likelihood that other types of tissue, such as muscle tissue, will experience some stimulation. Unwanted stimulation of muscle tissue, for example, may cause incidental cramps, stiffness, soreness or other side effects. These effects sometimes occur in the trapezius muscle given traditional stimulator placement below the skull region in contrast to the placement over the skull as described herein.

In some cases, a TENS (transcutaneous electrical nerve stimulator) unit may be used to pinpoint the location of the occipital nerve along the back of the head or on top of the head before the stimulator is implanted. This leads to improved stimulator placement proximate to the nerve being stimulated.

By way of further example, FIG. 7 shows an SCU (140) that has been implanted beneath the scalp of a patient. The SCU (140) may be implanted in a surgically-created shallow depression or opening in the skull (135). For instance, the depression may be made in the parietal bone, temporal bone, frontal bone, or any other bone within the skull (135) as best serves a particular application. The SCU (140) may conform to the profile of surrounding tissue(s) and/or bone(s), thereby minimizing the pressure applied to the skin or scalp.

As shown in FIG. 7, the lead (141) and/or catheter (143) run subcutaneously to the top of the skull (135) where stimulation can be optimally provided to the occipital nerves or other cranial nerves as described herein. This configuration provides the advantage of placing the SCU (104) at a surgically convenient location while still providing stimulation to the occipital nerves through the lead (141) in an environment with relatively low fat and soft tissue content so that the charging cycle the patient must endure is minimized.

With the stimulator optimally placed along the posterior or superior portion of the head a wide variety of headache and similar conditions can be treated through stimulation of the occipital nerves or other cranial nerves while minimizing the burden the charging cycle imposes on the patient. A general discussion of the types of headaches and related conditions that may be treated in this manner follows.

The International Headache Society (IHS) published “Classification and Diagnostic Criteria for Headache Disorders, Cranial Neuralgias and Facial Pain” in 1988. IHS identified 13 different general groupings of headache, given below in Table 1.

TABLE 1
Groupings of Headache Disorders and Facial Pain

| | |
|---|---|
| 1) | Migraine |
| 2) | Tension-type headache |
| 3) | Cluster headache and chronic paroxysmal hemicrania |
| 4) | Miscellaneous headaches unassociated with structural lesions |
| 5) | Headache associated with head trauma |
| 6) | Headache associated with vascular disorders |
| 7) | Headache associated with non-vascular intracranial disorder |
| 8) | Headache associated with substances or their withdrawal |
| 9) | Headache associated with non-cephalic infections |
| 10) | Headaches associated with metabolic disorders |
| 11) | Headache or facial pain associated with disorder of cranium, |
| | neck, eyes, ears, nose, sinuses, teeth, mouth or other facial |
| | or cranial structures |
| 12) | Cranial neuralgias, nerve trunk pain and deafferentation pain |
| 13) | Non-classifiable headache |

The IHS classification of the most common types of headache is summarized in Table 2 below.

TABLE 2
IHS Classification of Primary Headaches

| | |
|---|---|
| 1. | Migraine |
| 1.1 | Migraine without aura |
| 1.2 | Migraine with aura |
| 1.2.1 | Migraine with typical aura |
| 1.2.2 | Migraine with prolonged aura |
| 1.2.3 | Familial hemiplegic migraine headache |
| 1.2.4 | Basilar migraine |
| 1.2.5 | Migraine aura without headache |
| 1.2.6 | Migraine with acute onset aura |
| 1.3 | Ophthalmoplegic migraine |
| 1.4 | Retinal migraine |
| 1.5 | Childhood periodic syndromes that may be precursors to or |
| | associated with migraine |
| 1.5.1 | Benign paroxysmal vertigo of childhood |
| 1.5.2 | Alternating hemiplegia of childhood |
| 1.6 | Complications of migraine |
| 1.6.1 | Status migrainosus |
| 1.6.2 | Migrainous infarction |
| 1.7 | Migrainous disorder not fulfilling above criteria |
| 2. | Tension-type headache |
| 2.1 | Episodic tension-type headache |
| 2.1.1 | Episodic tension-type headache associated with disorder of |
| | pericranial muscles |
| 2.1.2 | Episodic tension-type headache not associated with disorder |
| | of pericranial muscles |
| 2.2 | Chronic tension-type headache |
| 2.2.1 | Chronic tension-type headache associated with disorder of |
| | pericranial muscles |
| 2.2.2 | Chronic tension-type headache not associated with disorder |
| | of pericranial muscles |
| 2.3 | Headache of the tension-type not fulfilling above criteria |
| 3. | Cluster headache and chronic paroxysmal hemicrania |
| 3.1 | Cluster Headache |
| 3.1.1 | Cluster headache, periodicity undetermined |
| 3.1.2 | Episodic cluster headache |
| 3.1.3. | Chronic Cluster Headache |
| 3.1.3.1 | Unremitting from onset |
| 3.1.3.2 | Evolved from episodic |
| 3.2 | Chronic paroxysmal hemicrania |
| 3.3 | Cluster headache-like disorder not fulfilling above Criteria |

Migraine Headache

The IHS classification provides diagnostic criteria for migraine without and with aura, summarized in Tables 3 and 4 below.

TABLE 3
IHS Diagnostic Criteria for Migraine Without Aura

| A. | At least five attacks fulfilling B-D below: | |
|---|---|---|
| B. | Headache attacks lasting 4-72 hours (untreated or unsuccessfully | |
| | treated) | |
| C. | Headache has at least two of the following characteristics: | |
| | 1. | Unilateral location |
| | 2. | Pulsating quality |
| | 3. | Moderate or severe intensity (inhibits or prohibits daily |
| | | activities) |
| | 4. | Aggravation by walking stairs or similar routine physical |
| | | activity |
| D. | During headache at least one of the following: | |
| | 1. | Nausea and/or vomiting |
| | 2. | Photophobia and phonophobia |
| E. | At least one of the following: | |
| | 1. | History and physical do not suggest headaches secondary to |
| | | organic or systemic metabolic disease |
| | 2. | History and/or physical and/or neurologic examinations do |
| | | suggest such disorder, but is ruled out by appropriate |
| | | investigations |
| | 3. | Such disorder is present, but migraine attacks do not occur |
| | | for the first time in close temporal relation to the disorder |

TABLE 4
IHS Diagnostic Criteria for Migraine With Aura

| A. | At least two attacks fulfilling B below: | |
|---|---|---|
| B. | At least three of the following four characteristics: | |
| | 1. | One or more fully reversible aura symptoms indicating focal |
| | | cerebral cortical and/or brain stem dysfunction |
| | 2. | At least one aura symptom develops gradually over more than |
| | | four minutes or two or more symptoms occur in succession |
| | 3. | No aura symptom lasts more than 60 minutes. If more than one |
| | | aura symptom is present, accepted duration is proportionally |
| | | increased |
| | 4. | Headache follows aura with a free interval of less than 60 |
| | | minutes. It may also begin before or simultaneously with |
| | | the aura. |
| C. | At least one of the following: | |
| | 1. | History and physical and neurologic examinations do not suggest |
| | | headaches secondary to organic or systemic metabolic disease |
| | 2. | History and/or physical and/or neurologic examinations do |
| | | suggest such disorder, but it is ruled out by appropriate |
| | | investigations |
| | 3. | Such disorder is present, but migraine attacks do not occur |
| | | for the first time in close temporal relation to the disorder |

The IHS classification includes several different types of migraine variants. Basilar migraine is defined as a migraine with an aura involving the brainstem. Symptoms include ataxia, dysarthria, vertigo, tinnitus and/or changes in consciousness and cognition. Ophthalmoplegic migraine is associated with acute attacks of third nerve palsy with accompanying dilation of the pupil. In this setting, the differential diagnosis includes an intracranial aneurysm or chronic sinusitis complicated by a mucocele. The ophthalmoplegia can last from hours to months. Hemiplegic migraine is distinguished by the accompanying hemiplegia, which can be part of the aura, or the headache may precede the onset of hemiplegia. Hemiplegic migraine can be familial and may last for days or weeks, clinically simulating a stroke. An additional differential diagnosis includes focal seizures.

Status migrainosus describes a migraine lasting longer than 72 hours with intractable debilitating pain, and typically occurs in a setting of inappropriate and prolonged use of abortive anti-migraine drugs. These patients may require hospitalization, both for pain control, detoxification from the abused drugs, and treatment of dehydration resulting from prolonged nausea and vomiting.

A migraine prevalence survey of American households was conducted in 1992, and included 20,468 respondents 12-80 years of age. Using a self-administered questionnaire based on modified IHS criteria, 17.6% of females and 5.7% of males were found to have one or more migraine headaches per year. A projection to the total US population suggests that 8.7 million females and 2.6 million males suffer from migraine headache with moderate to severe disability. Of these, 3.4 million females and 1.1 million males experience one or more attacks per month. Prevalence is highest between the ages of 25 and 55, during the peak productive years.

Based on published data, the Baltimore County Migraine Study, MEDSTAT's MarketScan medical claims data set, and statistics from the Census Bureau and the Bureau of Labor Statistics, it has been estimated that migraineurs require 3.8 bed rest days for men and 5.6 days for women each year, resulting in a total of 112 million bedridden days. Migraine costs American employers about $13 billion a year because of missed workdays and impaired work function—close to $8 billion is directly due to missed workdays. Patients of both sexes aged 30 to 49 years incurred higher indirect costs compared with younger or older employed patients. Annual direct medical costs for migraine care are about $1 billion, with about $100 spent per diagnosed patient. Physician office visits account for about 60% of all costs; in contrast, emergency department visits contribute less than 1% of the direct costs.

Tension-Type Headache

The diagnostic criteria for tension-type headaches are summarized in Table 5 below. However, migraine symptoms may overlap considerably with those of tension-type headaches. Tension-type headaches are believed by some experts to be a mild variant of migraine headache. Patients with tension-type headaches who also have migraines may experience nausea and vomiting with a tension headache, though when they do, it typically is mild and for a shorter duration compared to that with a migraine. Tension-type headache may be a disorder unto itself in individuals who do not have migraines, and may manifest as attacks of mild migraine in individuals with migraines.

TABLE 5
IHS Criteria for Various Forms of Tension-Type Headache

Tension-type headache
At least two of the following pain characteristics:

| | |
|---|---|
| 1. | Pressing/tightening (non-pulsating) quality |
| 2. | Mild or moderate intensity (may inhibit, but does not prohibit |
| | activities) |
| 3. | Bilateral location |
| 4. | No aggravation by walking stairs or similar routine physical |
| | activity |

Both of the following:

| | |
|---|---|
| 1. | No nausea or vomiting (anorexia may occur) |
| 2. | Photophobia and phonophobia absent, or only one is present |

At least one of the following:

| | |
|---|---|
| 1. | History and physical do not suggest headaches secondary to organic |
| | or systemic metabolic disease |
| 2. | History and/or physical and/or neurologic examinations do suggest |
| | such disorder, but is ruled out by appropriate investigations |
| 3. | Such disorder is present, but tension-type headache does not occur |
| | for the first time in close temporal relation to the disorder |

Episodic tension-type headache (ETTH)
Diagnostic criteria:

| | |
|---|---|
| A. | At least 10 previous episodes, <180 days/year (<15/mo) with |
| | headache |
| B. | Headache lasting from 30 minutes to 7 days |

Chronic tension-type headache (CTTH)
Diagnostic criteria:

| | |
|---|---|
| A. | Average frequency ≧ 1 day/month (≧189 days/year) for ≧ 6 |
| | months |

Tension-type headache associated with disorder of pericranial muscles
At least one of the following:

| | |
|---|---|
| 1. | Increased tenderness of pericranial muscles demonstrated by manual |
| | palpation or pressure algometer. |
| 2. | Increased electromyographic level of pericranial muscles at rest |
| | or during physiologic tests. |

Tension-type headache not associated with pericranial muscle disorder
No increased tenderness of pericranial muscles. If studied,
electromyography of pericranial muscles shows normal levels of
activity.

Based on a telephone survey of 13,345 people, the 1-year period prevalence of episodic tension-type headache (ETTH) is estimated to be 38.3%, according to IHS criteria. Women had a higher 1-year ETTH prevalence than men in all age, race, and education groups, with an overall prevalence ratio of 1.16. Prevalence peaked in the 30- to 39-year-old age group in both men (42.3%) and women (46.9%). Prevalence increased with increasing educational levels in both sexes, reaching a peak in subjects with graduate school educations of 48.5% for men and 48.9% for women. Of subjects with ETTH, 8.3% reported lost workdays because of their headaches, while 43.6% reported decreased effectiveness at work, home, or school.

Chronic Daily Headache

Chronic tension-type headache (CTTH) is a subtype of tension headaches, with patients experiencing headaches daily or almost every day. In practice, the term “chronic daily headache” is commonly used to describe headaches lasting for greater than 4 hours per day and for at least 15 days per month. The classification of chronic daily headaches is summarized below in Table 6.

TABLE 6
Classification of Chronic Daily Headache

Transformed migraine
1. With medication overuse
2. Without medication overuse
Chronic tension-type headache (CTTH)
1. With medication overuse
2. Without medication overuse
New daily persistent headache
1. With medication overuse
2. Without medication overuse
Hemicrania continua
1. With medication overuse
2. Without medication overuse

In the study of 13,345 people cited above, the 1-year period prevalence of chronic tension-type headache (CTTH) was estimated to be 2.2%. This prevalence was higher in women and declined with increasing education. Subjects with CTTH reported more lost workdays (mean of 27.4 days vs. 8.9 days for those reporting lost workdays) and reduced-effectiveness days (mean of 20.4 vs. 5.0 days for those reporting reduced effectiveness) compared with subjects with ETTH.

Chronic daily headaches are best conceptualized as an umbrella category term referring to a group of headache disorders characterized by headaches which occur greater than 15 days per month, with an average untreated duration of greater than 4 hours per day. There are many secondary causes of chronic daily headache, including post-traumatic headache, arthritis, intracranial mass lesions, etc. There are also short-lived primary headache disorders that occur greater than 15 days per month, such as chronic cluster headache or the paroxysmal hemicranias. The most common primary, chronic daily headache disorders include transformed migraine, chronic tension-type headaches, new daily persistent headache, or hemicrania continua. Each of these diagnoses can be complicated by medication overuse (e.g., barbiturates, acetaminophen, aspirin, caffeine, ergotamine tartrate and opioids). When used daily, all of these medications can lead to a vicious cycle of rebound headaches.

Cluster Headache

The 1988 IHS classification system recognized the uniqueness of cluster headache as a clinical and epidemiological entity. Formerly classified as a vascular migraine variant, cluster headache (a.k.a. suicide headache) is thought to be one of the most severe headache syndromes. It is characterized by attacks of severe pain, generally unilateral and orbital and lasting 15 minutes to 3 hours, with one or more symptoms such as unilateral rhinorrhea, nasal congestion, lacrimation, and conjunctival injection. In most patients, headaches occur in episodes, generally with a regular time pattern. These “cluster periods” last for weeks to months, separated by periods of remission lasting months to years. These headaches primarily affect men and in many cases patients having distinguishing facial, body, and psychological features. Several factors may precipitate cluster headaches, including histamine, nitroglycerin, alcohol, transition from rapid eye movement (REM) to non-REM sleep, circadian periodicity, environmental alterations, and change in the level of physical, emotional, or mental activity. The IHS classification system gives specific diagnostic criteria for cluster headache, as given in Table 7 below.

TABLE 7
IHS Diagnostic Criteria for Cluster Headache

3.1 Cluster Headache

| A. | At least 5 attacks fulfilling B-D below: | |
|---|---|---|
| B. | Severe unilateral, orbital, supraorbital and/or temporal pain lasting | |
| | 15-180 minutes untreated | |
| C. | At least one of the following signs present on the pain side: | |
| | 1. | Conjunctival injection |
| | 2. | Lacrimation |
| | 3. | Nasal congestion |
| | 4. | Rhinorrhea |
| | 5. | Forehead and facial sweating |
| | 6. | Miosis |
| | 7. | Ptosis |
| | 8. | Eyelid edema |
| D. | Frequency of attacks: from 1 every other day to 8 per day | |
| E. | At least one of the following: | |
| | 1. | History, physical and neurological examinations do not suggest |
| | | one of the disorders listed in groups 5-11 of Table 1 |
| | 2. | History and/or physical and/or neurological examinations do |
| | | suggest such disorder, but it is ruled out by appropriate |
| | | investigations |
| | 3. | Such disorder is present, but cluster headache does not occur |
| | | for the first time in close temporal relation to the disorder |

3.1.1 Cluster headache periodicity undefined

| | |
|---|---|
| A. | Criteria for 3.1 fulfilled |
| B. | Too early to classify as 3.1.2 or 3.1.3 |

3.1.2 Episodic cluster headache
Description: Attacks lasting between 1 week and 3 months occur in
periods lasting 1 week to one year separated by pain free periods
lasting 14 days or more.

| | |
|---|---|
| A. | All the letter headings of 3.1 |
| B. | At least 2 periods of headaches (cluster periods) lasting |
| | (untreated) from 7 days to one year, separated by remissions |
| | of at least 14 days. |

3.1.3 Chronic cluster headache
Description: Attacks lasting between 2 weeks and 3 months occur
for more than one year without remission or with remissions
lasting less than 14 days.

| | |
|---|---|
| A. | All the letter headings of 3.1 |
| B. | Absence of remission phases for one year or more or with |
| | remissions lasting less than 14 days. |

3.1.3.1 Chronic cluster headache unremitting from onset

| | |
|---|---|
| A. | All the letter headings of 3.1.3 |
| B. | Absence of remission periods lasting 14 days or more from |
| | onset. |

3.1.3.2 Chronic cluster headache evolved from episodic

| | |
|---|---|
| A. | All the letter headings of 3.1.3 |
| B. | At least one interim remission period lasting 14 days or |
| | more within one year after onset, followed by unremitting |
| | course for at least one year. |

The estimated prevalence of cluster headache is 69 cases per 100,000 people. Men are affected more commonly than women in a proportion of 6:1. Although most patients begin experiencing headache between the ages of 20 and 50 years (mean of 30 years), the syndrome may begin as early as the first decade and as late as the eighth decade.

Cervicogenic Headache

Cervicogenic headache (CEH) is a headache with its origin in the neck area. The source of pain is in structures around the neck that have been damaged. These structures can include joints, ligaments, muscles, and cervical discs, all of which have complex nerve endings. When these structures are damaged, the nerve endings send pain signals up the pathway from the upper nerves of the neck to the brainstem. These nerve fibers may synapse in the same brainstem nuclei as the nerve fibers of the trigeminal nerve. Since the trigeminal nerve is responsible for the perception of head pain, the patient experiences the symptoms of headache and/or facial pain.

While many patients who are diagnosed with CEH have the traditional symptoms of tension-type headache, some of the patients who have the traditional symptoms of migraine and cluster headache also respond to CEH diagnosis and treatment.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method of treating headaches comprising stimulating a nerve in a patient's head with an electrode implanted over the skull on a posterior or superior portion of the patient's head to alleviate headache pain.

2. The method of claim 1, wherein said stimulating a nerve comprises applying an electrical stimulation current to said nerve with said electrode.

3. The method of claim 1, further comprising implanting a stimulator, wherein said electrode is disposed on said stimulator and said stimulator is implanted over the skull on a posterior or superior portion of the patient's head.

4. The method of claim 1, further comprising implanting a stimulator in said patient that is connected to said electrode by a lead.

5. The method of claim 1, wherein said stimulating further comprises infusing one or more drugs into a stimulation site with a stimulator implanted with said electrode.

6. The method of claim 1, further comprising adjusting stimulation parameters that characterize a stimulus provided by said stimulator to optimize relief from said headache pain.

7. The method of claim 6, further comprising sensing at least one indicator related to said headaches and using said at least one sensed indicator to adjust one or more of said stimulation parameters.

8. The method of claim 7, wherein said at least one indicator includes any of electrical brain activity; neurotransmitter levels; hormone levels; metabolic brain activity; blood flow rate in the patient's head or neck and medication levels within the patient.

9. The method of claim 1, wherein said nerve is an occipital nerve.

10. The method of claim 1, further comprising selecting an implantation location for said electrode with a minimum of impedance due to fat and soft tissue, but still effective for stimulation of said nerve.

11. The method of claim 1, further comprising minimizing power requirements of an implanted stimulator connected to said electrode by placing said electrode in an implantation location with reduced impedance due to fat and soft tissue.

12. A method of treating headaches comprising stimulating a nerve in a patient's head with an electrode implanted at a location where impedance due to fat and soft tissue is minimized.

13. The method of claim 12, wherein said stimulating further comprises infusing one or more drugs into a stimulation site with an implanted stimulator connected to said electrode.

14. The method of claim 12, further comprising adjusting stimulation parameters that characterize a stimulus provided by said stimulator to optimize relief from said headache pain.

15. The method of claim 14, further comprising sensing at least one indicator related to said headaches and using said at least one sensed indicator to adjust one or more of said stimulation parameters.

16. The method of claim 15, wherein said at least one indicator includes any of electrical brain activity; neurotransmitter levels; hormone levels; metabolic brain activity; blood flow rate in the patient's head or neck and medication levels within the patient.

17. The method of claim 12, wherein said nerve is an occipital nerve.

18. The method of claim 12, further comprising implanting a stimulator at a surgically convenient location and directing a lead from said stimulator body to said electrode located where impedance due to fat and soft tissue is minimized.

19. A method of minimizing recharging cycles of an implanted stimulator, said method comprising:
minimizing power requirements of said implanted stimulator by stimulating a nerve in a patient's head at a location where electrical impedance due to fat and soft tissue is minimized to treat a condition of said patient;
wherein said minimized impedance increases an amount of time said stimulator can effectively operate between recharging cycles.

20. The method of claim 19, wherein said condition being treated comprises chronic headache pain.
